(19) United States
(12) Patent Application Publication
(10) Pub. No.: US 2024/0335673 A1
(43) Pub. Date: Oct. 10, 2024

(54) TREATMENT DEVICE AND CONTROL CIRCUIT THEREOF

(71) Applicant: SHENZHEN PENINSULA MEDICAL GROUP, Shenzhen (CN)

(72) Inventors: Yanan LI, Shenzhen (CN); Yongsheng LIANG, Shenzhen (CN); Xiaobing LEI, Shenzhen (CN); Sudi ZHANG, Shenzhen (CN)

(73) Assignee: SHENZHEN PENINSULA MEDICAL GROUP, Shenzhen (CN)

(21) Appl. No.: 18/648,755

(22) Filed: Apr. 29, 2024

Related U.S. Application Data
(63) Continuation of application No. PCT/CN2022/101640, filed on Jun. 27, 2022.

(30) Foreign Application Priority Data
Nov. 4, 2021 (CN) 202111302672.4

(51) Int. Cl. A61N 1/40

(52) U.S. Cl. CPC A61N 1/403

(57) ABSTRACT

Disclosed are a control circuit for controlling a treatment device and a treatment device. The control circuit for controlling the treatment device includes a radio frequency (RF) generating module, a low-frequency generating module, and a control assembly. The RF generating module and the low-frequency generating module are connected to the electrode sheet respectively. The control assembly is respectively connected to the RF generating module and the low-frequency generating module. The control assembly controls the RF generating module and the low-frequency generating module to output a RF signal and a low-frequency signal to the electrode sheet.

### CROSS-REFERENCE TO RELATED APPLICATIONS

The present application is a continuation of International Application No. PCT/CN2022/101640, filed on Jun. 27, 2022, which claims priority to Chinese Patent Application No. 202111302672.4, filed on Nov. 4, 2021, the entire contents of which are incorporated herein by reference.

### TECHNICAL FIELD

The present application relates to the technical field of medical devices, and in particular to a treatment device and a control circuit thereof.

### BACKGROUND

Radio frequency (RF) energy and low-frequency energy (low-frequency electrical stimulation) are used for treatment. By generating the thermal effect of biological tissue, the RF energy mainly promotes collagen regeneration, improves the elasticity and thickness of the connective tissue of the muscle layer and the elasticity of the fascia and ligaments, increases the ability to work with muscles, reduces fat cells, promotes blood circulation, improves the metabolism of inflammatory factors and pain-inducing factors, and relieves pain.

Low-frequency energy (low-frequency electrical stimulation) mainly acts on nerve cells, which can stimulate dormant nerve cells, restore related muscle functions and various reflexes, increase the number and speed of axon regeneration, improve related muscle functions, and promote the recovery of related reflexes.

However, the treatment effect of the single RF energy or low-frequency energy is poor.

### SUMMARY

The main purpose of the present application is to provide a control circuit for controlling a treatment device, aiming to combine the RF energy with the low-frequency energy to improve treatment effect of the treatment device.

In view of the above objectives, the control circuit for controlling a treatment device in the present application is used for outputting an electrical signal to at least one electrode sheet, including:
a radio frequency (RF) generating module, an output end of the RF generating module being connected to the at least one electrode sheet;
a low-frequency generating module, an output end of the low-frequency generating module being connected to the at least one electrode sheet; and
a control assembly, the control assembly being respectively connected to a controlled end of the RF generating module and a controlled end of the low-frequency generating module, and the control assembly being configured to control at least one of the RF generating module and the low-frequency generating module to output a RF signal and/or a low-frequency signal to the at least one electrode sheet.

In an embodiment, the at least one electrode sheet includes at least two electrode sheets, the control assembly includes:
a switch module, an input end of the switch module being respectively connected to the output end of the RF generating module and the output end of the low-frequency generating module, and an output end of the switch module being connected to the at least two electrode sheets; and
a first main controller, the first main controller being connected to a controlled end of the switch module, and the first main controller being configured to control the switch module to be on/off switchable, so as to control the RF generating module and the low-frequency generating module to alternately or simultaneously output the RF signal and the low-frequency signal to the at least two electrode sheets.

In an embodiment, the switch module includes:
a first electronic switch, an input end of the first electronic switch being connected to the output end of the low-frequency generating module, an output end of the first electronic switch being connected to the at least two electrode sheets, and a controlled end of the first electronic switch being connected to the first main controller; and
a second electronic switch, an input end of the second electronic switch being connected to the output end of the RF generating module, an output end of the second electronic switch being connected to the at least two electrode sheets, and a controlled end of the second electronic switch being connected to the first main controller.

In an embodiment, the control assembly further includes a temperature sensor connected to the first main controller, and the temperature sensor is configured to detect a temperature of a target area of a treatment object and output the temperature to the first main controller; and

the first main controller is further configured to control the first electronic switch to be on and the second electronic switch to be off in response to that the temperature of the target area rises to a first preset temperature, and the first main controller is further configured to control the first electronic switch to be off and the second electronic switch to be on in response to that the temperature of the target area drops to a second preset temperature.

In an embodiment, the control assembly further includes:
a timer connected to the first main controller;
in response to that the temperature of the target area rises to the first preset temperature, the first main controller is further configured to control the second electronic switch to be off, and then control the first electronic switch to be on after a first preset time lag by using the timer; and/or
in response to that the temperature of the target area drops to the second preset temperature, the first main controller is further configured to control the first electronic switch to be off, and then control the second electronic switch to be on after a second preset time lag.

In an embodiment, the first main controller is further configured to control the RF generating module and the low-frequency generating module to stop working after the RF generating module and the low-frequency generating module being working for a third preset time.

In an embodiment, the at least two electrode sheets are divided into a plurality of groups, and the plurality of groups of the at least two electrode sheets are arrayed in the target area;
the control assembly is further configured to control the RF generating module to output the RF signal to each group of the at least two electrode sheets sequentially until the target area reaches the first preset temperature; and/or
the control assembly is further configured to control the low-frequency generating module to output the low-frequency signal to each group of the at least two electrode sheets sequentially until the target area drops to the second preset temperature.

In an embodiment, the control assembly further includes:
a low-pass filter, one end of the low-pass filter being connected to the output end of the low-frequency generating module, and the other end of the low-pass filter being connected to the at least two electrode sheets;
a high-pass filter, one end of the high-pass filter being connected to the output end of the RF generating module, and the other end of the high-pass filter being connected to the at least two electrode sheets; and
a second main controller configured to control the RF generating module and the low-frequency generating module to work.

In an embodiment, the RF generating module is a high-frequency power supply configured to generate the RF signal, and a frequency of the RF signal is in a range from 200 kilohertz to 50 MHz.

In an embodiment, the low-frequency generating module is a low-frequency power supply configured to generate the low-frequency signal, and a frequency of the low-frequency signal is in a range from 800 Hz to 1200 Hz.

In an embodiment, the control assembly is an integrated microcontroller or a digital signal processor.

In an embodiment, the first main controller is a microcontroller or a digital signal processor.

In an embodiment, the switch module is an electronic switch array composed of plurality of electronic switches.

In an embodiment, a number of the electronic switches is equal to a number of the at least two electrode sheets.

In an embodiment, a number of the first electronic switches is equal to a number of output ends of the low-frequency generating module, and a number of the second electronic switches is equal to a number of output ends of the RF generating module.

In an embodiment, the first electronic switch includes at least one selecting from a group of a triodes, a metal-oxide-semiconductor tube, and an insulated gate bipolar transistor.

In an embodiment, the first preset temperature is in a range from 46° C. to 50° C. and the second preset temperature is in a range from 36° C. to 40° C.

In an embodiment, both the first preset time lag and the second preset time lag are in a range from 0 to 2 seconds.

The present application further provides a treatment device including:
a treatment device body; and
a treatment tip, being detachably connected to the treatment device body;
at least one electrode sheet being provided on the treatment tip; and
the control circuit as mentioned above being provided in the treatment device body.

In the technical solution of the present application, the control assembly is configured to control the RF generating module and the low-frequency generating module to output RF signals and low-frequency signals to the electrode sheet, so that the RF signals and low-frequency signals are outputted to the target area through the electrode sheet. In this way, the RF signals and low-frequency signals are combined to treat the target area, thereby effectively improving the treatment effect.

### BRIEF DESCRIPTION OF THE DRAWINGS

To more clearly illustrate technical solutions in the embodiments of the present application or the related art, the following will briefly introduce the drawings that need to be used in the description of the embodiments or the related art. Obviously, the drawings in the following description are only some embodiments of the present application. For those skilled in the art, without creative effort, other drawings can be obtained according to the structures shown in these drawings.

FIG. 1 is a structural schematic diagram of a treatment tip of a treatment device according to an embodiment of the present application.

FIG. 2 is a circuit diagram of a control circuit for controlling a treatment device according to an embodiment of the present application.

FIG. 3 is a circuit diagram of the control circuit for controlling the treatment device according to another embodiment of the present application.

FIG. 4 is a circuit diagram of the control circuit for controlling the treatment device according to yet another embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art based on the embodiments of the present application without creative efforts shall fall within the claimed scope of the present application.

It should be noted that all the directional indications (such as up, down, left, right, front, rear . . . ) in the embodiments of the present application are only used to explain the relative positional relationship, movement, or the like of the components in a certain posture (as shown in the drawings). If the specific posture changes, the directional indication will change accordingly.

Besides, the descriptions associated with, e.g., “first” and “second,” in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. Further, if “and/or” appears throughout the text, it includes three parallel schemes. Taking “A and/or B” as an example, it includes the scheme A, or the scheme B, or the scheme that the scheme A and the scheme B satisfy at the same time. In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

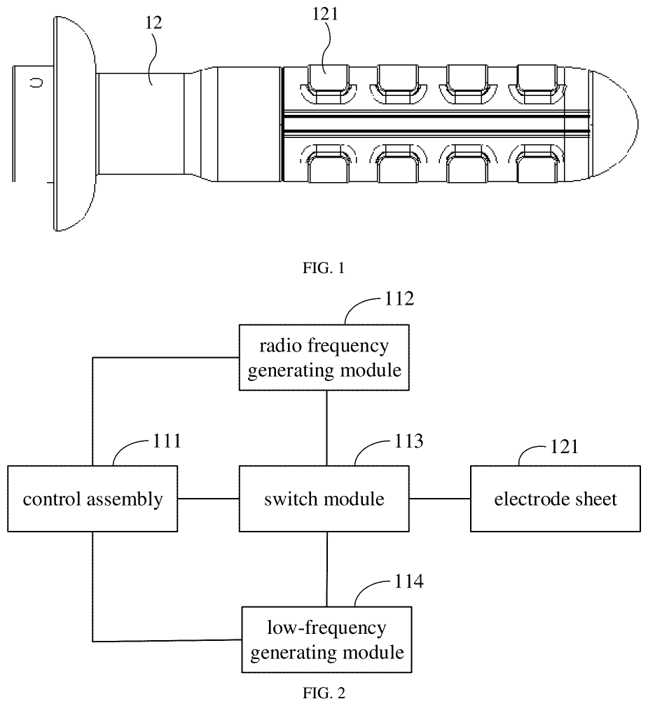

As shown in FIG. 1, the present application proposes a control circuit for controlling a treatment device. The control circuit for controlling the treatment device can be applied to a treatment device. The treatment device may include a treatment device body and a treatment tip 12. The treatment tip 12 may be provided with at least one electrode sheet 121. There may be one electrode sheet (monopolar) or two electrode sheets 121 (bipolar with a positive electrode and a negative electrode) or more than two electrode sheets 121. The electrode sheet 121 is connected to the target area A. The target area A may be the biological tissue, such as the pelvic floor muscle.

The control circuit for controlling the treatment device outputs radio frequency (RF) signals and low-frequency signals to the electrode sheet 121, thereby achieving treatment of the target area A. Through the thermal effect of biological tissue, the applied RF signal can promote collagen regeneration, improve the elasticity and thickness of the connective tissue of the muscle layer and the elasticity of the fascia and ligaments, increase the ability of the pelvic floor muscles to work together, promote blood circulation, improve the metabolism of inflammatory factors and pain-inducing factors, and relieve pain. The applied low-frequency signal can act on nerve cells, tp stimulate dormant nerve cells, restore related muscle functions and various reflexes, increase the number and speed of axon regeneration, improve related muscle functions, and promote the recovery of related reflexes.

As shown in FIG. 2, in an embodiment of the present application, the treatment control circuit includes:

a RF generating module 112, an output end of the RF generating module 112 being connected to the electrode sheet 121;

a low-frequency generating module 114, an output end of the low-frequency generating module 114 being connected to the electrode sheet 121; and

a control assembly 111, the control assembly 111 being respectively connected to a controlled end of the RF generating module 112 and a controlled end of the low-frequency generating module 114, and the control assembly 111 being configured to control the RF generating module 112 and the low-frequency generating module 114 to work to output a RF signal and a low-frequency signal to the electrode sheet 121.

In this embodiment, the RF generating module 112 can be a high-frequency power supply or other instrument that can generate RF electrical signals. The RF signal can be the high-frequency current or other RF signals, and the frequency of the high-frequency current can range from 200 kilohertz to 50 MHz, such as 10 MHZ.

The low-frequency generating module 114 can be a low-frequency power supply or other instrument that can generate a low-frequency electrical signal. The low-frequency signal can be the low-frequency current or other low-frequency signals. The frequency of the low-frequency current can range from 800 Hz to 1200 Hz, such as 1000 Hz.

The control assembly 111 can be an integrated microcontroller, a digital signal processor or other controller. The control assembly 111 controls the RF generating module 112 and the low-frequency generating module 114. That is, the control assembly 111 can control the RF generating module 112 and the low-frequency generating module 114 to work alternately, or to work simultaneously, or the control assembly 111 can control one of the RF generating module 112 and the low-frequency generating module 114 to work and the other of the RF generating module 112 and the low-frequency generating module 114 to stop working. Or, the control assembly 111 can further control the RF generating module 112 and the low-frequency generating module 114 to work continuously at the same time, and the switch is provided at the output end of the low-frequency generating module 114 and the output end of the RF generating module 112 respectively. By controlling the switch to be on/off, RF signals and low-frequency signals are outputted to the electrode sheet 121 alternately, or RF signals and low-frequency signals are outputted to the electrode sheet 121 simultaneously, or only RF signals are outputted to the electrode sheet 121, or only low-frequency signals are outputted to the electrode sheet 121.

In the technical solution of the present application, the control assembly 111 is configured to control the RF generating module 112 and the low-frequency generating module 114 to output RF signals and low-frequency signals to the electrode sheet 121, so that the RF signals and low-frequency signals are outputted to the target area A through the electrode sheet 121. In this way, the RF signals and low-frequency signals are combined to treat the target area A, thereby effectively improving the treatment effect.

As shown in FIG. 2, in an embodiment, the control assembly 111 includes:

a switch module 113, an input end of the switch module 113 being respectively connected to an output end of the RF generating module 112 and an output end of the low-frequency generating module 114, and an output end of the switch module 113 being connected to the electrode sheet 121; and

a first main controller, the first main controller being connected to a controlled end of the switch module 113, and the first main controller is configured to control the switch module 113 to be on and off alternately, to control the RF generating module 112 and the low-frequency generating module 114 to alternately output signals to the electrode sheet 121.

In this embodiment, the first main controller can be a microcontroller, a digital signal processor or other controllers.

The switch module 113 may be an electronic switch array composed/integrated of plurality of electronic switches. The input ends of the plurality of electronic switches are the input ends of the switch module 113, and the output ends of the plurality of electronic switches are the output ends of the switch module 113. The controlled ends of the plurality of electronic switches are the controlled ends of the switch module 113. The number of the electronic switches may be equal to the number of output ends of the RF generating module 112 and output ends of the low-frequency generating module 114, or may be equal to the number/logarithm of the electrode sheets 121. The first main controller controls the RF generating module 112 and the low-frequency generating module 114 to start and continue to work, and controls the plurality of electronic switches to be on/off by outputting high-level and low-level control signals, thereby outputting RF signals and low-frequency signals to the electrode sheets 121 alternately.

In some embodiments, the switch module 113 may further be a switching switch. The input end of the switching switch is the input end of the switch module 113. The output end of the switching switch is the output end of the switch module 113, and the controlled end of the switching switch is the controlled end of the switch module 113. The input ends of the switching switch can be divided into two groups, which are respectively connected to the output end of the RF generating module 112 and the output end of the low-frequency generating module 114. The number of the output ends of the switching switch can be equal to the number/logarithm of the electrode sheets 121. The first main controller controls the RF generating module 112 and the low-frequency generating module 114 to start and continue to work, and controls the two groups of input ends of the switching switch to be connected to the output end of the RF generating module 112 and the output end of the low-frequency generating module 114 alternately by outputting high-level and low-level control signals, thereby outputting the RF signals and the low-frequency signals to the electrode sheets 121 alternately.

In this embodiment, by setting the switch module 113, only one of the RF generating module 112 or the low-frequency generating module 114 is connected to the electrode sheet 121 at the same time, that is, the RF signal and the low-frequency signal are alternately outputted to the electrode sheet 121 at different time, which can effectively prevent the RF generating module 112 and the low-frequency generating module 114 from being connected to the electrode sheet 121 at the same time, causing the low-frequency signal to flow to the RF generating module 112 through the electrode sheet 121, and the RF signal to flow to the low-frequency generating module 114 through the electrode sheet 121, thereby avoiding interfering with the work of the RF generating module 112 and the low-frequency generating module 114, and causing the output signals of the RF generating module 112 and the low-frequency generating module 114 to be abnormal or even damaged. In this way, the crosstalk between the low-frequency signal and the RF signal can be effectively avoided.

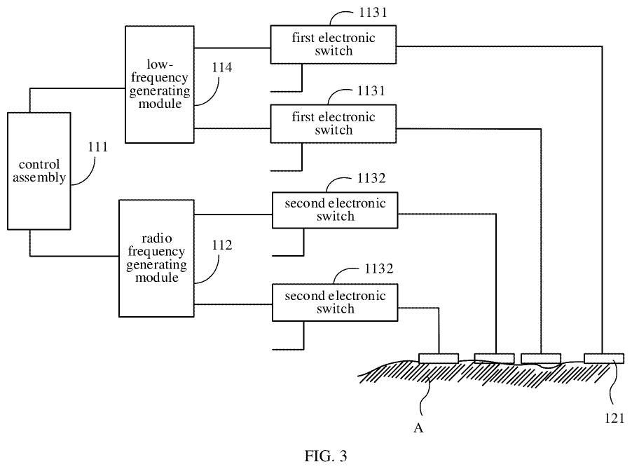

As shown in FIG. 3, in an embodiment, the switch module 113 includes: a first electronic switch 1131, an input end of the first electronic switch 1131 being connected to the output end of the low-frequency generating module 114, an output end of the first electronic switch 1131 being connected to the electrode sheet 121, and a controlled end of the first electronic switch 1131 being connected to the first main controller; and

a second electronic switch 1132, an input end of the second electronic switch 1132 being connected to the output end of the RF generating module 112, an output end of the second electronic switch 1132 being connected to the electrode sheet 121, and a controlled end of the second electronic switch 1132 being connected to the first main controller.

The number of the first electronic switches 1131 can be one or more, and in some embodiments, the number of the first electronic switches 1131 can be equal to the number of the output ends of the low-frequency generating module 114. The number of the second electronic switches 1132 can be one or more, and in some embodiments, the number of the second electronic switches 1132 can be equal to the number of the output ends of the RF generating module 112.

The first electronic switch 1131 and/or the second electronic switch 1132 may be one or more combinations of triodes, metal-oxide-semiconductor (MOS) tubes, and insulated gate bipolar transistors (IGBTs).

In this embodiment, by setting plurality of electronic switches as the first electronic switch 1131 and the second electronic switch 1132, the first main controller can control one or more first electronic switches 1131 to be on at the same time to output one or more low-frequency signals, control one or more second electronic switches 1132 to be on at the same time to output one or more RF signals, and alternately control the first electronic switch 1131 and the second electronic switch 1132 to be on to output RF signals and low-frequency signals alternately.

As shown in FIG. 3, in an embodiment, the control assembly 111 further include:

a temperature sensor connected to the first main controller, and the temperature sensor is configured to detect a temperature of a target area of a treatment object and output the temperature.

The first main controller is further configured to control the first electronic switch 1131 to be on and the second electronic switch 1132 to be off in response to that the temperature of the target area rises to a first preset temperature.

The first main controller is further configured to control the first electronic switch 1131 to be off and the second electronic switch 1132 to be on in response to that the temperature of the target area drops to a second preset temperature.

In this embodiment, the temperature sensor can be set on or near the electrode sheet 121. When the treatment device is working, the electrode sheet 121 can contact the biological tissue of the target area A and collect the temperature of the biological tissue.

The RF signal can heat up the target area A through the thermal effect of the biological tissue, and when it switches to the low-frequency signal, the biological tissue begins to cool down. In this embodiment, the output power of the RF signal can range from 5 watts to 30 watts, to avoid too fast temperature rise. In this embodiment, by detecting the temperature of biological tissue as a standard for switching between RF signals and low-frequency signals, an exact switching signal is provided, which can not only effectively improve the accuracy of switching between RF signals and low-frequency signals, and achieve good combined treatment, but also can avoid burns caused by excessive temperature of biological tissue.

In some embodiments, the first preset temperature can range from 46° C. to 50° C., such as 48° C.

The second preset temperature can range from 36° C. to 40° C., such as 38° C. That is, under the control of the control circuit for controlling the treatment device, the target temperature ranges between 38° C. and 48° C., so that the skin tissue will not be burned while the treatment effect is achieved.

As shown in FIGS. 1 to 3, in an embodiment, the control assembly 111 further includes:

a timer connected to the first main controller.

In response to that the temperature of the target area A rises to the first preset temperature, the first main controller is further configured to control the second electronic switch 1132 to be off and control the first electronic switch 1131 to be on after a first preset time lag by using the timer; and/or

in response to that the temperature of the target area A drops to the second preset temperature, the first main controller is further configured to control the first electronic switch 1131 to be off and control the second electronic switch 1132 to be on after a second preset time lag.

The first preset time lag may be the same as or different from the second preset time lag, which may be set according to actual needs. In this embodiment, the first preset time lag may be the same as the second preset time lag, and both the first preset time lag and the second preset time lag may range from 0 to 2 seconds, such as 1 second. The timer completes the timing of the first time lag and the second time lag.

In this embodiment, when the RF signal is switched to the low-frequency signal, the first preset time is delayed by reserving the first preset time. And/or, when the low-frequency signal is switched to the RF signal, the second preset time or the second preset time is delayed. Thus, it can be fully avoided that when the RF signal is switched to the low-frequency signal, the RF generating module 112 and the low-frequency generating module 114 are simultaneously connected to the electrode sheet 121 at a certain moment, resulting in crosstalk between the RF signal and the low-frequency signal.

In one embodiment, the first main control is further configured to control the RF generating module 112 and the low-frequency generating module 114 to stop working after the RF generating module 112 and the low-frequency generating module 114 have been working for the third preset time.

In this embodiment, the third preset time can be a preset treatment time. When the RF generating module 112 and the low-frequency generating module 114 alternately output signals to the electrode sheet 121 and the treatment time of the target area A reaches the preset treatment time, the RF generating module 112 and the low-frequency generating module 114 are controlled to stop working, thereby realizing automatic control of the treatment device, avoiding too long treatment time, avoiding over-treating the target area A, and avoiding causing damage to the biological tissue of the target area A.

As shown in FIG. 1, there are plurality groups of electrode sheets 121, and the plurality groups of electrode sheets 121 are distributed in the target area A sequentially. For example, there are 4 groups of electrode sheets 121, and there are 2 sheets in each group.

The control assembly 111 is further configured to control the RF generating module 112 to sequentially output RF signals to each group of electrode sheets 121 until the target area A reaches the first preset temperature; and/or

the control assembly 111 is further configured to control the low-frequency generating module 114 to sequentially output low-frequency signals to each group of electrode sheets 121 until the target area A drops to the second preset temperature.

In this embodiment, there may be plurality groups of electrode sheets 121, and the plurality groups of electrode sheets 121 are sequentially distributed in the target area A. The plurality groups of electrode sheets 121 are cyclically heated, and the temperature of the target area corresponding to each group of electrode sheets is monitored. When the temperature of the target area reaches the preset value, the RF signal is switched to the next group of electrode sheets. The target area A rises to the first preset temperature synchronously.

Compared with the method that only two larger electrode sheets 121 are set, in the present application, the large area of the target area A is heated, and the energy uniformity is improved.

Compared with the method that plurality groups of electrode sheets 121 are heated at the same time, in this embodiment, the temperature of each group of electrode sheets 121 is detected and is controlled separately, to avoid different heating degrees due to different path resistance values of different groups of electrode sheets 121, which may cause some electrode sheets 121 to have too high temperatures and burn the human body while some electrode sheets 121 cannot reach the target temperature.

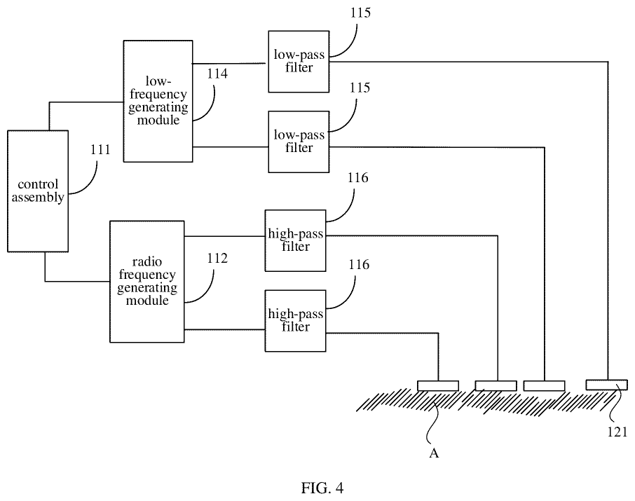

As shown in FIG. 4, in an embodiment, the control assembly 111 further includes:
a low-pass filter 115, one end of the low-pass filter 115 being connected to the output end of the low-frequency generating module 114, and the other end of the low-pass filter 115 being connected to the electrode sheet 121;
a high-pass filter 116, one end of the high-pass filter 116 being connected to the output end of the RF generating module 112, and the other end of the high-pass filter 116 being connected to the electrode sheet 121; and
a second main controller configured to control the RF generating module 112 and the low-frequency generating module 114 to work.

The second main controller and the first main controller mentioned above can be the same controller, or they can be set separately.

The low-pass filter can be an integrated filter or a filter circuit built by discrete components. The bandwidth of the low-pass filter can be determined according to the bandwidth of the low-frequency signal outputted by the low-frequency generating module, ensuring that the low-frequency signal can pass through the low-pass filter while the RF signal cannot pass through the low-pass filter.

The high-pass filter can be an integrated filter or a filter circuit built by discrete components. The bandwidth of the high-pass filter can be determined according to the bandwidth of the RF signal outputted by the RF generating module, ensuring that the RF signal can pass through the high-pass filter while the low-frequency signal cannot pass through the high-pass filter.

In this embodiment, by setting the low-pass filter 115, the low-frequency signal outputted by the low-frequency generating module 114 can pass through the low-pass filter 115 smoothly and flow to the electrode sheet 121, and the RF signal can be filtered out to avoid the RF signal from interfering with the low-frequency generating module 114. The high-pass filter 116 is provided to enable the RF signal outputted by the RF generating module 112 pass through the high-pass filter 116 smoothly and flow to the electrode sheet 121, and the low-frequency signal can be filtered out to avoid the low-frequency signal from interfering with the RF generating module 112.

In this embodiment, not only RF signals and low-frequency signals can be outputted simultaneously to the electrode sheet 121 for treatment, but also there is no crosstalk between the low-frequency signal and the RF signal.

In order to better illustrate the technical principle of the present application, the control method of an embodiment of the present application will be described below in combination with the above embodiments.

S100, when the treatment device is started, entering the RF mode, outputting the RF signal to the electrode sheet 121, and monitoring the temperature of the target area A in real time through the temperature sensor.

The RF mode can refer to controlling the RF generating module 112 to work and the low-frequency generating module 114 not to work, or it can refer to controlling the RF generating module 112 and the low-frequency generating module 114 to work simultaneously, and controlling the switch module 113, so that only the RF signal of the RF generating module 112 can be outputted to the electrode sheet 121.

S200, when the temperature of the target area A rises to the first preset temperature, switching to the low-frequency mode and outputting the low-frequency signal to the electrode sheet 121.

The low-frequency mode can refer to controlling the low-frequency generating module 114 to work and the RF generating module 112 not to work, or it can refer to controlling the low-frequency generating module 114 and the RF generating module 112 to work simultaneously, and controlling the switch module 113, so that only the low-frequency signal of the low-frequency generating module 114 can be outputted to the electrode sheet 121.

S300, when the temperature of the target area A drops back to the second preset temperature, switching to the RF mode, and outputting the RF signal to the electrode sheet 121.

The first preset temperature and the second preset temperature are the preset temperature ranges when the treatment device is working, which can avoid burns and cam achieve the best treatment effect.

S400, repeating steps S200 and S300 until the start time of the treatment device reaches the third preset time (preset treatment duration), and controlling the treatment device to shut down.

In addition, in some embodiments, when the RF mode is switched to the low-frequency mode, the first preset time can be reserved to avoid the RF signal and the low-frequency signal being outputted to the electrode sheet 121 simultaneously during switching and causing crosstalk. When the low-frequency mode is switched to the RF mode, the second preset time can be reserved to avoid the RF signal and the low-frequency signal being outputted to the electrode sheet 121 simultaneously during switching and causing crosstalk.

The present application further proposes a treatment device, which includes a treatment device body and a treatment tip 12. The electrode sheet 121 is provided at the treatment tip 12.

The treatment device body is provided with the control circuit for controlling the treatment device as mentioned above. The specific structure of the control circuit for controlling the treatment device can refer to the above embodiments. Since the treatment device adopts all the technical solutions of all the above embodiments, it has at least all the beneficial effects brought by the technical solutions of the above embodiments, which will not be repeated here.

Further, the treatment device body is detachably connected to the treatment tip 12.

Thus, after using the treatment tip 12 or after the treatment tip 12 is damaged, a new treatment tip 12 can be replaced to avoid replacing the entire treatment device, thereby effectively reducing costs, and improving the hygiene of the treatment device.

The above are only some embodiments of the present application, and do not limit the scope of the present application thereto. Under the concept of this application, any equivalent structural transformation made according to the description and drawings of the present application, or direct/indirect application in other related technical fields shall fall within the claimed scope of the present application.

1-10. (canceled)

11. A control circuit for controlling a treatment device to output an electrical signal to at least one electrode sheet, comprising:
a radio frequency (RF) generating module, wherein an output end of the RF generating module is connected to the at least one electrode sheet;
a low-frequency generating module, wherein an output end of the low-frequency generating module is connected to the at least one electrode sheet; and
a control assembly, wherein the control assembly is respectively connected to a controlled end of the RF generating module and a controlled end of the low-frequency generating module, and the control assembly is configured to control at least one of the RF generating module and the low-frequency generating module to output a RF signal and/or a low-frequency signal to the at least one electrode sheet.

12. The control circuit according to claim 11, wherein the at least one electrode sheet comprises at least two electrode sheets, the control assembly comprises:
a switch module, wherein an input end of the switch module is respectively connected to the output end of the RF generating module and the output end of the low-frequency generating module, and an output end of the switch module is connected to the at least two electrode sheets; and
a first main controller, wherein the first main controller is connected to a controlled end of the switch module, and the first main controller is configured to control the switch module to be on/off switchable, so as to control the RF generating module and the low-frequency generating module to alternately or simultaneously output the RF signal and the low-frequency signal to the at least two electrode sheets.

13. The control circuit according to claim 12, wherein the switch module comprises:
a first electronic switch, wherein an input end of the first electronic switch is connected to the output end of the low-frequency generating module, an output end of the first electronic switch is connected to the at least two electrode sheets, and a controlled end of the first electronic switch is connected to the first main controller; and
a second electronic switch, wherein an input end of the second electronic switch is connected to the output end of the RF generating module, an output end of the second electronic switch is connected to the at least two electrode sheets, and a controlled end of the second electronic switch is connected to the first main controller.

14. The control circuit according to claim 13, wherein the control assembly further comprises:
a temperature sensor connected to the first main controller, and the temperature sensor is configured to detect a temperature of a target area of a treatment object and output the temperature to the first main controller; and
the first main controller is further configured to control the first electronic switch to be on and the second electronic switch to be off in response to that the temperature of the target area rises to a first preset temperature, and the first main controller is further configured to control the first electronic switch to be off and the second electronic switch to be on in response to that the temperature of the target area drops to a second preset temperature.

15. The control circuit according to claim 14, wherein the control assembly further comprises:
a timer connected to the first main controller;
in response to that the temperature of the target area rises to the first preset temperature, the first main controller is further configured to control the second electronic switch to be off, and then control the first electronic switch to be on after a first preset time lag by using the timer; and/or
in response to that the temperature of the target area drops to the second preset temperature, the first main controller is further configured to control the first electronic switch to be off, and then control the second electronic switch to be on after a second preset time lag.

16. The control circuit according to claim 15, wherein the first main controller is further configured to control the RF generating module and the low-frequency generating module to stop working after the RF generating module and the low-frequency generating module being working for a third preset time.

17. The control circuit according to claim 13, wherein: the at least two electrode sheets are divided into a plurality of groups, and the plurality of groups of the at least two electrode sheets are arrayed in the target area;
the control assembly is further configured to control the RF generating module to output the RF signal to each group of the at least two electrode sheets sequentially until the target area reaches the first preset temperature; and/or
the control assembly is further configured to control the low-frequency generating module to output the low-frequency signal to each group of the at least two electrode sheets sequentially until the target area drops to the second preset temperature.

18. The control circuit according to claim 11, wherein the control assembly further comprises:
a low-pass filter, wherein one end of the low-pass filter is connected to the output end of the low-frequency generating module, and the other end of the low-pass filter is connected to the at least two electrode sheets;
a high-pass filter, wherein one end of the high-pass filter is connected to the output end of the RF generating module, and the other end of the high-pass filter is connected to the at least two electrode sheets; and
a second main controller configured to control the RF generating module and the low-frequency generating module to work.

19. The control circuit according to claim 11, wherein the RF generating module is a high-frequency power supply configured to generate the RF signal, and a frequency of the RF signal is in a range from 200 kilohertz to 50 MHz.

20. The control circuit according to claim 11, wherein the low-frequency generating module is a low-frequency power supply configured to generate the low-frequency signal, and a frequency of the low-frequency signal is in a range from 800 Hz to 1200 Hz.

21. The control circuit according to claim 11, wherein the control assembly is an integrated microcontroller or a digital signal processor.

22. The control circuit according to claim 12, wherein the first main controller is a microcontroller or a digital signal processor.

23. The control circuit according to claim 12, wherein the switch module is an electronic switch array composed of plurality of electronic switches.

24. The control circuit according to claim 23, wherein a number of the electronic switches is equal to a number of the at least two electrode sheets.

25. The control circuit according to claim 13, wherein a number of the first electronic switches is equal to a number of output ends of the low-frequency generating module, and a number of the second electronic switches is equal to a number of output ends of the RF generating module.

26. The control circuit according to claim 13, wherein the first electronic switch comprises at least one selecting from a group of a triodes, a metal-oxide-semiconductor tube, and an insulated gate bipolar transistor.

27. The control circuit according to claim 14, wherein the first preset temperature is in a range from 46° C. to 50° C. and the second preset temperature is in a range from 36° C. to 40° C.

28. The control circuit according to claim 15, wherein both the first preset time lag and the second preset time lag are in a range from 0 to 2 seconds.

29. A treatment device, comprising:
a treatment device body; and
a treatment tip detachably connected to the treatment device body;
at least one electrode sheet provided on the treatment tip; and
the control circuit according to claim 11 provided in the treatment device body.
